(19) United States
(12) Patent Application Publication
(10) Pub. No.: US 2025/0186000 A1
(43) Pub. Date: Jun. 12, 2025

(54) METHOD FOR GENERATING AN EXTRACT OF SIGNIFICANT PASSAGES OF A VIDEO FOOTAGE

(71) Applicant: SPARKBIO S.R.L., San Lazzaro di Savena (IT)

(72) Inventor: Sebastiano Zannoli, San Lazzaro di Savena (IT)

(21) Appl. No.: 18/843,831

(22) PCT Filed: Mar. 16, 2023
(86) PCT No.: PCT/IB2023/052570

§ 371 Date: Sep. 4, 2024

(30) Foreign Application Priority Data
Mar. 16, 2022 (IT) 102022000005063

(51) Int. Cl. A61B 5/00; G16H 40/67

(52) U.S. Cl. CPC A61B 5/743; A61B 5/7435; G16H 40/67

(57) ABSTRACT

A method for generating an extract of significant steps of a video footage representative of the performance of a surgical operation or complex diagnostic investigation is produced by a medical recorder 100 and displayed on its monitor. And includes:
the definition of a set of markers provided in the video footage, and representative of the beginning and end of significant passages thereof;
the identification, in said video footage, of one or more pairs of said markers, each of said pairs being constituted by an initial marker and a final marker defining portions;
merging all the recorded portions of the video footage in order to obtain, on the computer medium, a significant extract of the whole video footage.

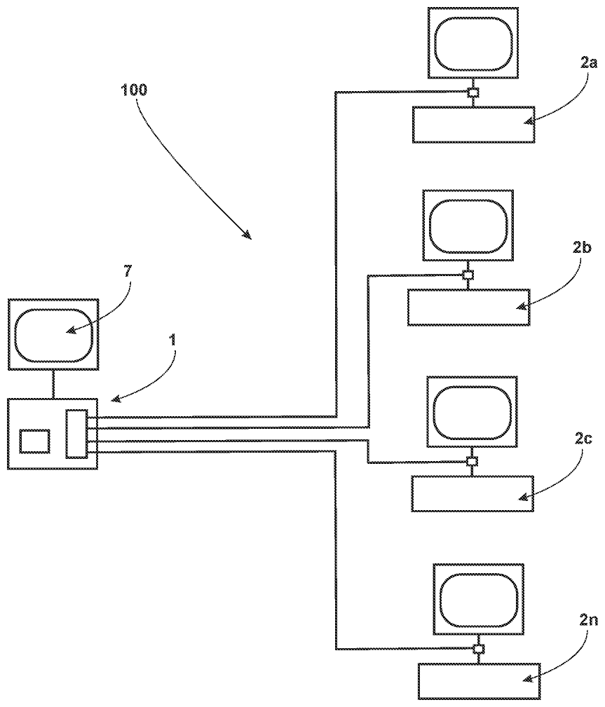

### TECHNICAL FIELD

The present invention relates to the field of electromedical equipment for monitoring the physiological parameters of patients undergoing surgical operation or diagnostic investigations; in particular, it applies to a system for collecting, displaying, and recording, on a computer medium, the synchronized signals coming from different instruments and equipment, used by the operators for therapeutic purposes or for monitoring the surgical operation or diagnostic investigation.

### BACKGROUND ART

As is known, during a surgical operation, there are many apparatuses in the operating room having different functions, for example, a surgical robot, an angiographic arch aimed at carrying out a radiological investigation, a polygraph or multiparameter monitor which allows to display different signals which correspond, for example, to an electrocardiogram or blood pressure, or an echo-Doppler apparatus or device programmers, used to define the functionality of devices installed on the patients body during the procedure or after the surgical operation, for example, a pacemaker or an external ventricular assist pump. In addition, one or more environment video cameras are usually provided in the operating room, in order to record the surgical operation from different angles so as to document its performance for educational or more generally scientific purposes.

Each of the above-mentioned apparatuses generates its own set of information, which is related to one or more parameters related to the patient's condition, and in particular to the trend of the physiological parameters that must be kept under constant control during surgical operation. This information, which is used to control certain functions, is generally represented graphically, transformed into representative numerical values, displayed in the form of constantly updated images or video footages, and stored in storage media inside the separate devices or otherwise via devices connected thereto.

For standardization purposes, the information is transformed into suitable formats, such as those known by the acronyms “DICOM” or “HL7”, which allow the information to be easily stored in large memory banks, also shared among all the equipment.

### Technical Problem

This operation makes it possible to form many separate files collected in a single large memory device but does not allow to check the value or trend of a parameter when a certain event occurs, based on information obtained from another apparatus.

In more explicit terms and by way of example, it is particularly useful for the surgeon or for those who are in charge of monitoring the patient's condition, to see, for example, how the situation detected by the ultrasound image evolves at the same time as the electrocardiogram trend. Again, by way of example, if the electrocardiogram shows a certain pathological event involving the heart, it may be useful, for the purpose of determining the cause of the event, to verify a posteriod, through the archive, the patency condition at that precise moment of the cardiac muscle's blood perfusion system through the evaluation of the corresponding angiographic image obtained by the radiological system.

Another need that is evident in the operating room, but also in other areas where complex diagnostic investigations are performed, is to control the various parameters and significant physiological data of the patient, taken from the various instruments, at the same time, as they are being acquired, with the possibility of seeing and evaluating these parameters and data at the same time in a single display, and to possibly perform operations on them to highlight particular situations, always keeping in mind the moment in which they occurred.

There are known systems that allow the acquisition of signals from the analog main or auxiliary outputs of one or more electromedical instruments, in a specialized computer, to store and process the same signals and provide useful information on them.

A significant progress in the ability to integrate information from a set of electromedical instruments and equipment into a single monitor that is immediately accessible to the operator, and above all to synchronize and subsequently manage that information together, has been achieved with the invention described in the International Patent Application published under number WO2016/092503A1, by the same Applicant (see US 2018/0263574 A1).

That invention discloses a system for the combined acquisition and displaying of output signals from a set of different electromedical instruments and equipment, and for the synchronized storage of the signals.

The above-mentioned signals are related to the trend of corresponding physiological parameters of a patient, or to images or films recorded during a diagnostic examination or surgical operation performed by an operator—usually a surgeon, but in some cases also a specialized technician—or by a team of operators.

In particular, the system comprises video signal or data line derivative devices, each of which is installed in the output connection section of a respective instrument or equipment with its display units, to make available the video or data outputs.

The derived signals converge towards a central signal collection and processing unit, consisting of a dedicated computer in which a manager program operates. The latter includes procedures for the acquisition and transcoding the signals, procedures for the combined displaying thereof in a single monitor available to the operator, for the introduction of time references aimed at identifying significant situations and events of the diagnostic examination procedure or surgical operation in progress, and procedures for storing the combined signals, including synchronization and time reference information. The program also includes operator interface and input/output procedures designed to manage a sufficient number of input information channels so as to integrate and synchronize all the most important instruments and equipment used in the most common diagnostic and interventional procedures performed in the operating room.

The system described above allows the operator carrying out the diagnostic examination or surgical operation, first of all, to see all the information coming from the equipment and instruments in the room on a single screen, whether in the form of images or video footage or in the form of strings of numerical data; secondly, it allows him to enter time references (“markers”) at situations of interest, or top events, of the surgical operation or examination in progress, and to synchronize these references with all the information detected for that given diagnostic examination or surgery operation.

In this way, the operator can see all the information useful for the ongoing operation on a single monitor in the form of windows of different sizes representing the video outputs of certain apparatuses, for example, a surgical robot and an angiographic arch, and at the same time information in the form of alphanumeric strings from other apparatuses, for example, the physiological parameters received from the multiparameter polygraph (systolic and diastolic pressure, oxygen saturation, etc.).

The display of this information can be manipulated, that is the windows representing the information can be enlarged to bring it to the foreground and into a central area of the monitor, if in a given stage of the operation in progress it is particularly interesting to observe; otherwise, the information can be reduced and displayed in a peripheral area of the monitor, if in that stage the information from another apparatus is more important.

The possibility of entering in every moment a time reference, which is associated to all the information acquired in that moment, provides the operator with an additional tool to find and visualize top moments of the operation later on, since the operator has all significant video and/or text information immediately available. This makes it easy to find all the information relating to the above-mentioned top moments, both during the operation itself and later on, when the operation is over. In particular, this feature is particularly advantageous if the operator is interested in analyzing these moments in detail, or if the operator intends to use the operation footage for teaching or information purposes, for example during a lecture or a presentation at a specialized conference or seminar.

The systems described above help the surgical team to facilitate the visualization on a single monitor of the different information generated by the medical instrumentation and the visualization devices on the surgical field, and they make it possible to obtain a complete and synchronized digital recording of the entire surgical operation and the video and audio information that was displayed during it.

In the context of documenting a particularly significant surgical operation or complex diagnostic investigation, the sequential recording of the events and instrumental data acquires particular importance with regard to the didactic and, more generally, informative aspects, for which the aforesaid surgical operation may be particularly useful.

In fact, the surgeon who performs the surgical operation is very often interested to use the recording of the surgical operation, for instance during a congress or a lecture, to better describe and illustrate its course and the medical-surgical techniques that have been used. Since a surgical operation can last up to several hours, it is clear that reproducing the entire recording is hardly ever advantageous or feasible.

In such cases, the surgeon usually extracts a summary of the salient phases of the surgical operation from the complete recording, which lasts a few minutes or in any case for a time available for the exposure. Extracting the summary requires the work of searching for interesting material and extracting and assembling it into a new file of shorter duration, in one or more post-production sessions that can take a long time.

With the pace of work and other professional and extra-professional commitments, the great difficulty for a distinguished surgeon to find time for these operations is self evident and in any case, other more important and productive activities can be affected by that.

### OBJECTS OF THE INVENTION

The main object of the present invention is to propose a method for the extraction of significant phases of a video footage relating to the monitoring of a surgical operation or a complex diagnostic investigation and its storage in digital form.

A further object of the invention is to propose a method capable of performing such an extraction in real time, during the above-mentioned surgical operation.

Another object of the invention is to propose a method capable of performing this extraction from the complete recording of the above-mentioned surgical operation.

### SUMMARY OF THE INVENTION

These and other objects will be achieved by the method proposed by the present invention, carried out in accordance with the contents of the independent claim. Further objects are achieved by means of the features and variations of the method claimed in the dependent claims.

The invention comprises a method for generating an extract of significant steps of a video footage representative of the performance of a surgical operation or complex diagnostic investigation produced by a medical recorder and displayed on its monitor.

The method is implemented by a processor selection program and comprises the execution of the following operations on the video footage, being displayed and recorded or already recorded:
the definition of a set of markers provided in the above-mentioned video footage, and representative of the beginning and end of significant passages of the same;
the identification, in said video footage, of one or more pairs of said markers, each of said pairs being constituted by an initial marker and a final marker, corresponding to the beginning and the end of a respective significant passage of said video footage;
in correspondence with each initial marker, the starting of duplication of the portion of the video footage included between the initial marker and the corresponding final marker and the recording of the portion of video footage on a computer medium, independent from the registration of the whole video footage;
merging all the recorded portions of the video footage in order to obtain a significant extract of the whole video footage on a computer readable storage medium.

### BRIEF DESCRIPTION OF THE DRAWING

The characteristics of the invention which do not appear from what has been said before, are pointed out in the following description, with reference to the enclosed drawing, in which:
The sole FIGURE represents a simplified block diagram of a system for the combined synchronisation and displaying of information, improved by the invention.

### DESCRIPTION OF PREFERRED EMBODIMENTS OF THE INVENTION

With reference to the sole FIGURE, a system for the combined acquisition, synchronization and display of data and information from the monitoring of a diagnostic investigation or surgical operation is illustrated. In particular, the system comprises a medical recorder 100, the essential characteristics and functions of which are known from the above-described International Patent Application published at WO2016/092503A1. This document is referred to for further details relating to the structure and operation of the medical recorder 100.

The medical recorder 100 allows to acquire, synchronize and continuously view, on one monitor 7, video or data output signals from a set of instruments and apparatuses 2a, 2b, 2c, . . . 2n, normally used in an operating room during a surgical operation or a diagnostic investigation, for example an angiography, performed on a patient. A central unit 1 of the recorder 100 also records the entire sequence of frames displayed on the monitor 7 in one or more digital video files on a suitable non-transitory computer readable medium, for example a hard disc, a solid state device (SSD) memory device, or another device with which the medical recorder 100 is provided.

In addition to the instruments and apparatuses 2a, 2b, 2c, . . . 2n, one or more environment video cameras, not illustrated in the FIGURE, are connected to the medical recorder 100 and are intended to register continuously the operating field, from certain angles of view intended to provide exhaustive documentation of the operations carried out during the surgical operation.

An audio channel, also connected to central unit 1, comprises a microphone and the analog-digital circuitry necessary to acquire and digitize a signal generated by the microphone and to make it available for subsequent processing. The microphone is preferably available to the surgeon performing the operation and recording his or her voice.

By way of example, and not by way of limitation, it is specified that the set of different instruments and apparatuses 2a, 2b, 2c, . . . 2n may include, inter alia, a surgical robot, an angiographic arch, and/or a polygraph or multiparameter monitor, and/or a thoracic strain-gauge, and/or an echo-Doppler control panel, X-ray equipment, an IUVS device (apparatus for intravascular ultrasound), and/or a set of medical device programmers, and all equipment for anaesthesia and respiratory monitoring and support. Obviously, the list is not exhaustive and in any case only some of the listed instruments and equipment may be provided.

A suitable processing program, residing and operating in the central unit 1, combines the obtained digital images into a single image, to obtain corresponding composite images, in which two or more of these digital images are suitably arranged in the combined image, according to viewing scenarios defined by an operator. The combined image is then reproduced on the monitor 7, which is connected with or integrated into the above-mentioned central unit 1.

The above described operation is repeated continuously for each frame acquired at the same moment by the different acquisition channels, in order to obtain, on the monitor 7, a continuous video footage in real time showing the footages taken by the apparatuses or instruments 2a, 2b, 2c, . . . 2n selected in the currently defined scenario. A procedure for changing scenario allows the operator to change the displayed scenario at any time and to replace it with different scenarios that may have been previously or currently defined.

Similarly, the audio stream generated by the microphone, containing the surgeon's speech, is recorded on the same file or a separate one.

The monitor 7 can display, in addition to the above described video signals, also data signals, either analog or digital, from instruments which provide analog or values indicative of an amplitude of the monitored physiological parameter.

A remote control, not illustrated in the FIGURE, likewise connected to the central unit 1, is intended to provide the processing program with signalling or command information from an operator, for example the surgeon performing the surgical operation. The function of the remote control will be explained in more detail below.

Advantageously, the processing program that manages the system functions in the central unit 1 further comprises a procedure for inserting time “markers” at images relating to significant events of an operating session. Such markers may be entered by an operator via usual input interfaces provided in the central unit 1, for example a mouse or keyboard, either when the significant event occurs or in a later post-acquisition step.

For the purposes of the present invention, particular markers are entered into the video footage via the above-mentioned remote control. Substantially, the surgeon who considers that the operation has reached a significant phase can signal the beginning and end of this significant phase by pushing special buttons provided in the remote control and sending corresponding signals to the central unit 1.

The processing program further comprises a procedure for generating video files in a standard format (for example, .AVI format), beginning from the combined frame sequences, for the scenario displayed on the monitor 7 in every moment, and for the entire duration of the diagnostic investigation or surgical operation.

The method according to the invention comprises operations for automatically generating an extract of significant steps of the above-mentioned video footage.

This method arises from the need of the surgeon performing the operation, which may last up to several hours, to obtain an extract of the video footage of the surgical operation, lasting from a few minutes to a few tens of minutes, which contains the most significant steps of the surgical operation. The length is generally a function of the importance of the surgical operation and the use of the extract, for example, a presentation at a congress or during a lecture.

According to the conventional way of obtaining the extract, the surgeon, using special video processing tools, reviews the entire video footage and ‘cuts’ the excerpts that he or she considers most interesting, then assembles them manually into a new video sequence, which will be stored as a single file independent of the original video footage. This procedure, as already mentioned, is time-consuming for the surgeon.

According to a preferred embodiment of the invention, selection means operating in the central unit 1 of the medical recorder 100, are constituted by a suitable computer program, which comprise procedures for scanning the video footage, identifying a set of markers provided in the original video footage or in other files synchronized therewith, extracting significant steps on the basis of said markers and recording these significant steps in a nontransitory computer readable medium.

In particular, the above-mentioned markers are identified by pairs, namely a start marker and an end marker of the sequence, are representative of the start and end of the aforesaid significant steps, and therefore mark the parts of the original video footage to be extracted and stored separately.

The set of markers according to the invention may be defined in advance, before the surgical operation begins, by the surgeon or another operator of the operating room, by means of a procedure integrated into the selector program, which interfaces with it by means of the user interface provided in the medical recorder 100.

One type of marker advantageously usable for the purposes of the invention is a marker derived from the audio signal acquired during surgical operation and synchronized with the corresponding video signal of the surgical operation recording. Substantially, the surgeon is instructed to speak with a certain continuity only when he or she considers that the phase of the surgical operation he or she is performing is significant and should be included in the extract of the video footage. A suitable procedure of the selector program identifies, by means of an algorithm based on the exceeding of predefined audio intensity thresholds, the moments in which the surgeon begins to speak and those in which he or she stops speaking. The first moments constitute an initial marker, while the second ones constitute a final marker. The definition of the latter interrupts the vocal flow for more than an established period of time, in order to avoid misinterpreting a simple pause in speech, intentional or unintentional, as a moment in which the speech is completed.

Another type of marker is given by particular icons or symbols that appear in predetermined areas of the video image, generated by one or more of the medical apparatuses or instruments whose outputs contribute to forming the video footage of the surgical operation. For this purpose, a further procedure of the aforesaid selector program is intended to perform a scan on selected frames of the video footage to detect the presence of such icons or symbols in the frames, and to associate, for example, an initial marker to the appearance of a particular icon or symbol, and a final marker to its disappearance.

By way of example, the appearance of an icon can be detected in the frames of the video footage, signalling the activation of the radiographic equipment that produces real-time X-ray images of the operating field. In fact, it is expected that the phases of the surgical operation that require a radiological view of the operating field by the surgical team should be considered particularly significant and are worthy of being included in the extract of the video footage.

Other ways of defining the markers can be found according to particular needs, without however going beyond the scope of the invention.

During the surgical operation, in accordance with the extraction method as envisaged by the invention, the selector program scans the video stream and possibly the audio stream synchronized therewith in order to find the above-mentioned markers defined previously, and organizes them by pairs of markers, initial and final, respectively. Therefore, these pairs of markers identify as many significant steps of the video footage to be included in the extract to be carried out.

At this point, the extraction phase of the significant steps can be carried out, for example, in real time and in a completely automatic way, by the selector program, duplicating the portions of the video footage and possibly of the audio stream, included between each initial marker and the subsequent final marker, and merging in a sequence the various portions of the video footage and storing them to form a single file of the extract of the significant steps, possibly keeping the pairs of markers to delimit the portions of the recording. The surgeon can review subsequently the extract to see whether or not to keep all the recorded portions, or to delete the ones that are considered unnecessary.

According to a variant of the method, the selector program identifies and enters the marker pairs into a stored copy of the complete video footage. At a later stage, the selector program can replay the entire video footage to the surgeon or another operator in charge, making it possible to position the visualization at the portions of the video footage identified by the different marker pairs, and making it possible to keep them, eliminate them from the extract, or add some parts of the video footage not identified by the program as significant, but considered as such by the surgeon. The surgeon may then instruct the selector program to remove from the extract those parts of the video footage that are considered insignificant after the review.

In this case, the selector program may advantageously propose to the surgeon the possibility of adding one or more parts of the original video footage, which are considered less significant, to the extract, in an accelerated form, in such a way as to guarantee a visual continuity between different significant portions of the footage, without however extending excessively the total duration of the extract.

According to an alternative embodiment of the invention, the above-mentioned selector program operates in a computer external to the medical recorder 100. In this case, the working steps of the method remain the same as those already described for the preferred embodiment and are carried out on a file containing the complete video footage, taken from the one stored in the central unit 1 of the medical recorder 100 and stored on a nontransitory computer readable medium of the external computer.

The advantages of implementing the extraction method according to the above-described invention are quite evident. Firstly, the surgeon is provided with a pre-packaged extract of the significant parts of the video footage relating to the performed surgical operation, which does not force the surgeon to go through lengthy sessions of viewing and selection of such parts but requires only a brief final review.

Another advantage derives from the fact that the surgeon can customize the structure of the extract in post-processing, either by adding portions of the video footage that he considers appropriate to the extract, or by removing portions that he considers unnecessary for the purpose of the extract.

A further advantage derives from the fact that different versions of the extract can be prepared in post-processing depending on the particular requirements of different uses.

It is understood that the described method can also be applied, with appropriate variations that are in any case within the inventive concept in question, to other areas and fields, without departing from the spirit of the invention.

It is also understood that different embodiments or variations of the present method, even if not explicitly described, are considered to fall within the scope of protection granted to the present invention as described above and defined in the following claims.

1. Method for generating an extract of significant passages of a video footage, said video footage representative of performance of a surgical operation or complex diagnostic investigation, said video footage carried out by a medical recorder (100), displayed on a monitor (7) and recorded on a non-transitory computer readable medium residing in a central unit (1) of said medical recorder (100), and further comprising combined displaying and recording of a video signal of at least one environment video camera framing an operating field and information and data from a plurality of medical instruments or apparatuses (2a, 2b, 2c, . . . 2n) to form a recorded entirety of the performance of the surgical operation or complex diagnostic investigation; said method implemented by selection means, comprising at least one selection computer program, operating in said central unit (1) of said medical recorder (100) or in another data processing unit external thereto, and including execution, on said video footage, while displayed and recorded or already recorded on said non-transitory computer readable medium, of the following operations:
defining a set of markers present in the video footage, and representative of moments of beginning and end of significant passages of the surgical operation or complex diagnostic investigation;
identifying, in said video footage, of one or more pairs of said markers, each of said pairs constituted by an initial marker and a final marker, respectively corresponding to moments of the beginning and end of a relevant cited significant passage of said video footage representative of performance of said surgical operation or complex diagnostic investigation;
in correspondence with each said initial marker, recording the corresponding portion of said video footage included between said initial marker and the corresponding final marker and wherein the recording of said portion of video footage on said non-transitory computer readable medium is independent of the recorded entirety of the performance of the surgical operation or complex diagnostic investigation; and
merging said recorded portions of the video footage to obtain said extract of significant Passages of the video footage.

2. The method according to claim 1, wherein the identifying of said marker pairs is performed by a surgeon, or by another person in charge, during the Performance of the surgical operation or complex diagnostic investigation, by sending commands to said central unit (1) by means of a remote control connected thereto.

3. The method according to claim 1, wherein the identifying of said marker pairs is performed by a procedure for recognizing predefined icons or symbols, belonging to said selector program and operating on frames of said video footage, displayed on said monitor (7) of said medical recorder (100), in order to detect presence in or disappearance from said frames of said predefined icons or symbols for association with corresponding markers.

4. The method according to claim 3, wherein said predefined icons or symbols are sought in parts of said frames corresponding to representation of video outputs of particular medical-diagnostic equipment used in an operating room and connected to said medical recorder (100).

5. The method according to claim 1, further comprising an audio signal relating to a voice of a surgeon performing the surgical operation or complex diagnostic investigation, wherein in said set of markers, the initial markers are defined by when the surgeon begins to speak, and the final markers are defined by when the surgeon stops speaking.

6. The method according to claim 1, further comprising capturing portions of said video footage not included between said pairs of markers and inserting said captured portions at an accelerated playback speed into said video footage extract.

7. The method according to claim 1, further comprising reviewing said video footage extract, manually performed by an operator to confirm presence or elimination of one or more of said recorded portions.
